(19) United States
(12) Patent Application Publication
(10) Pub. No.: US 2023/0018283 A1
(43) Pub. Date: Jan. 19, 2023

(54) HAIR GROWTH COMPOSITIONS

(71) Applicant: Apex Energetics Incorporated, Irvine, CA (US)

(72) Inventor: Michael Galitzer, MD, Los Angeles, CA (US)

(21) Appl. No.: 17/864,329

(22) Filed: Jul. 13, 2022

Related U.S. Application Data
(60) Provisional application No. 63/221,311, filed on Jul. 13, 2021.

(51) Int. Cl. A61K 36/76; A61K 36/185; A61K 31/7105; A61K 33/42; A61K 35/36

(52) U.S. Cl. CPC A61K 36/76; A61K 36/185; A61K 31/7105; A61K 33/42; A61K 35/36

(57) ABSTRACT

A hair growth composition comprising Betula Pubescens Flower Bud, Populus Nigra Leaf Bud, at least one human hair, and RNA. The hair growth composition may also comprise Castanea Vesca and phosphorus. An effective amount of the hair growth composition can be administered sublingually, topically, by thin film patch, in tablet form, or a combination thereof.

### CROSS-REFERENCE TO RELATED APPLICATIONS

This application claims the benefit of U.S. Provisional Ser. No. 63/221,311 filed on Jul. 13, 2021, which is incorporated by reference as if fully restated herein.

### FIELD OF THE DISCLOSURE

The present disclosure relates hair growth compositions to grow hair and/or prevent hair loss in mammals, particularly in humans that can be applied at least sublingually and/or topically.

### BACKGROUND OF THE DISCLOSURE

Hair loss, also referred to in its various identifications as male/female-pattern baldness and alopecia areata, is a universal occurrence found in men and women of all ages. The early signs of hair loss begin with a receding hairline or loss of hair at the crown. It is often those areas of the hair will get shorter or thinner and the hair follicle will produce shorter, finer hairs in a process called miniaturization. The follicle will inevitably stop producing hair and the hair loss will become obvious. The average individual loses anywhere between 50 to 100 hairs each day. This loss of hair begins at an elevated rate for one-in-three men aged between mid-teens to late twenties and the loss of hair becomes noticeable in two-thirds of American men by the age of 35. By the age of 50, 85% of men will lose a significant amount of their hair.

Dealing with hair loss has severe psychological effects that lead to several concerning mental health issues, including but not limited to, depression, anxiety, social phobia, lack of interest or pleasure in activities, loss of energy, sleep deprivation, difficulty controlling emotions, heightened tensions, and suicidal thoughts.

There are different composition on the market for treatments of hair loss however they fail to create a sustainable, efficient, and functional solution to the hair loss issue. The current treatments for hair re-growth have an assortment of harmful side effects. Some of the topical treatments include symptoms of developing acne at the site of application, burning of the scalp, increased facial hair growth, inflammation, or soreness at the root of follicles, a reddening of the skin, swelling of the face and other unwanted symptoms.

The use of these treatments can be difficult to manage because a balance of quantity and consistent application is inevitably inconsistent and may result in overuse leading to thinning of the skin or worsening of hair loss. Current hair growth compositions require ersonal application of such treatment is required one to two times daily and for an indefinite period of time to continue maintenance of hair growth. It is inconvenient and unmanageable to see a professional for application on every use and requires a degree of personal expertise and exhaustible diligence. Known topical growth compositions have a tendency to make hair look dirty and/or greasy and must be applied multiple times a day.

Many injections for hair loss treatment result in constipation, diarrhea, or issues resulting in an upset stomach. Additionally, certain fields of injectable treatments attempt block testosterone conversion leading to sexual issues of erectile dysfunction and other hormonal related side effects.

Other alternatives for hair loss treatment include congestible methods taken orally and have been widely known not to improve hair growth in generally healthy hair, rather only show effectiveness in a small percentage of users with certain conditions that are causing the hair loss. No clinical studies to date have shown certain congestible methods of hair growth to improve unhealthy hair in reversing dryness, shine, thickness, and other facets of common hair loss symptoms.

Ideally, it would be extremely beneficial to be able to have ease of portion control, consistency, and effectiveness with hair re-growth without the difficulty of inconsistent application, prevention of daily use, or symptomatic trade-offs with advantages to uses.

Accordingly, there is need for a solution to at least one of the problems. For example, there is an established need for a preferably convenient remedy that isn't restricted to a certain category of user experiencing hair loss and that promotes hair re-growth, shine, dryness, and thinning of hair without requiring the use of injection, topical application, or unreliable results with only temporary lasting effects.

### SUMMARY OF THE DISCLOSURE

The hair growth compositions disclosed herein comprises active ingredients comprising Betula Pubescens Flower Bud, Populus Nigra Leaf Bud, human hair, and RNA that is combined with inactive ingredients consisting of alcohol and water to be administered for topical use, sublingual use, or a combination of both. The hair growth composition may also comprise Castanea Vesca and phosphorus.

Accordingly, in one aspect, the present application relates to a hair growth composition comprising Betula Pubescens Flower Bud, Human hair, Populus Nigra Leaf Bud, and RNA.

Accordingly, in one aspect, the present application relates to a hair growth composition comprising Betula Pubescens Flower Bud, Castanea Vesca, Human hair, Phosphorous, Populus Nigra Leaf Bud, and RNA.

Accordingly, in one aspect, the present application relates to a hair growth composition comprising Betula Pubescens Flower Bud, Castanea Vesca, Human hair, Phosphorous, Populus Nigra Leaf Bud, RNA, and at least one inactive ingredient.

Accordingly, in one aspect, the present application relates to a hair growth composition comprising Betula Pubescens Flower Bud, Castanea Vesca, Human hair, Phosphorous, Populus Nigra Leaf Bud, RNA, and at least one inactive ingredient. The inactive ingredient can be alcohol, water, or a combination thereof.

In another aspect, the present application relates to a method of topically administering the hair growth composition to a subject in need thereof. The hair growth composition can be applied to the skin of the head, face, or body, or location where hair is desired to grow

In another aspect, the present application relates to a method of administering the hair growth composition to a subject in need thereof comprising sublingually administering a hair regrowth composition of the present application by the subject.

The hair growth compositions of the present application have improved hair regrowth in men and women who are experiencing hair loss. The hair growth composition, when applied topically or sublingually, can provide relief from hair loss, itchy scalp, and/or scaliness of scalp. The RNA may be extracted from fungus.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

The present disclosure is directed, in part, to a hair growth composition comprising an active ingredient portion and an inactive ingredient. The active ingredient portion comprises at least one essential element chosen from the group consisting of a human hair, a Betula Pubescens Flower Bud, a Castanea Vesca, a phosphorous, a Populus Nigra Leaf Bud, and an RNA, or any combination thereof. The active ingredient solution may be combined with an inactive ingredient such as alcohol, water, or a combination thereof to create a solution for treatment and/or prevention of hair loss as well as relief from itchy scalp, and scaliness of scalp. The hair growth composition can be applied or administered topically, sublingually, or in a combination thereof.

The compositions of the present disclosure can comprise or consist essentially of the essential elements and limitations of the disclosure described herein, as well any of the additional or optional ingredients, components, or limitations described herein or would be known to be added by a person having ordinary skill in the art.

The term “comprising” (and its grammatical variations) as used herein is used in the inclusive sense of “having” or “including” and not in the exclusive sense of “consisting only of” The terms “a” and “the” as used herein are understood to encompass the plural as well as the singular.

The term “effective amount” as used herein means an amount of a compound or composition such as a topical or system active sufficient to significantly induce a positive benefit, for example, hair growth or regrowth, but low enough to avoid serious side effects, i.e., to provide a reasonable benefit to risk ratio, within the scope of sound judgment of the skilled artisan.

The term “administered” as used herein means administration of a therapeutically effective amount of one or more compositions of the application to a cell, tissue, organ, or subject. Administration may take place sublingually or topically.

The essential elements in active ingredient solution in the hair growth composition comprise quantities of the human hair, the Betula Pubescens Flower Bud, the Populus Nigra Leaf Bud, and the RNA, measured by the Centisemal scale, known as the C-scale. The hair growth composition may also comprise quantities of phosphorous and Castanea Vesca, measured by the Centisemal scale. The quantities may be diluted quantities. The C scale, common in homeopathic solutions, signifies that a substance was diluted by a factor of 100 at each stage and X signifies a decimal dilution scale diluted by a factor of 10 at each stage.

The active ingredient solution may contain diluted Centisemal levels from 1X to 3X, 2X-3X , or 1X-2X of Betula Pubescens Flower Bud. In a further embodiment, the active ingredient solution may contain diluted Centisemal levels of 1×, 2X , or 3X of Betula Pubescens Flower Bud.

The Betula Pubescens Flower Bud may comprise of sesquiterpenoids, triterpenoids, flavonoids, and phenylpropenoids compounds. The sesquiterpenoid compounds may comprise of β-caryophyllene, birkenal, 6-hydroxy-β-caryophyllene, 6-hydroxy-β-caryophyllene acetate, trace-14-hydroxy-β-caryophyllene, 14-hydroxy-β-isocaryophyllene and/or 14-hydroxy-β-caryophyllene acetate compounds. The triterpenoid compounds may comprise of dammaradien-3-one, dipterocarpol lupen-20(29)-en-28-al and/or betulinic acid compounds. The flavonoid compounds may comprise of apigenin, sakuranetin, kumatakenin, 3′-methoxyapigenin, rhamnocitrin, pectolinaringenin, cirsimaritin, and/or catechin compounds. The phenylpropenoids compounds may comprise of 6-hydroxycaryophyllene p-coumarate, 14-hydroxycaryophyllene p-coumarate, 6-hydroxycaryophyllene ferulate, 14-hydroxycaryophyllene caffeate, and/or n-docosyl p-coumarate compounds. The Betula Pubescens Flower Bud may also contain quantities of Aliphatic C16-C28 acids and esters, Aliphatic C20-C28 alcohols, and/or Alkanes.

The active ingredient solution may further contain diluted centisemal levels from 1X to 3X, 2X-3X , or 1X-2X of Castenea Vesca. In a further embodiment, the active ingredient solution may contain diluted centisemal levels of 1×, 2X , or 3X of Castenea Vesca.

The active ingredient solution may further contain diluted Centisemal levels from 3C to 8C, 4C to 8C, 5C to 8C, 6C to 8C, 7C to 8C, 3C to 7C, 4C-7C, or 3C-6C of human hair. In a further embodiment, the active ingredient solution may contain diluted Centisemal levels of 4C and/or 7C of human hair. At least one intact strand of human hair can be soaked in a high-proof alcohol to prepare the at least one strand of human hair to be added to the composition.

The active ingredient solution may further contain diluted Centisemal levels from 4X to 9×, 11X-12×, 2C-7C of Phosphorous. In a further embodiment, the active ingredient solution may contain diluted Centisemal levels of 5×, 6×, 8×, 12×, 3C, 5C, 6C, and 7C of Phosphorous.

The active ingredient solution may further contain diluted Centisemal levels from 1X to 3X, 2X-3X , or 1X to 2X of Populus Nigra Leaf Bud. In a further embodiment, the active ingredient solution may contain diluted Centisemal levels of 1×, 2X , or 3X of Populus Nigra Leaf Bud.

The active ingredient solution may further contain diluted Centisemal levels from 2X to 5×, 3X to 5X , or 4X-5X of RNA. In a further embodiment, the active ingredient solution may contain diluted Centisemal levels of 2X, 3X and 4X of RNA.

The active ingredient solution may combine with the inactive ingredients. The inactive ingredients may be an alcohol-based and/or water ingredient. In a further embodiment, the alcohol ingredient may be 5% to 30% by volume of the hair growth composition and the water may be distilled. The alcohol ingredient in the hair growth composition may be 5% to 20% by volume. The alcohol ingredient may be 10% to 25%, 12% to 25%, 14%-25%, or 15%-25% by volume. The alcohol ingredient may be 17%, 18%, 19%, 20%, 21%, 22%, 23%, 24% or 25% by volume.

In hair loss treatment in accordance with the disclosure, an effective amount of said essential elements will be such an amount which will at least decrease the rate of hair loss or at times even prevent further loss of hair or induce hair regeneration. Namely, an effective amount of said active ingredient solution given within the framework of a hair loss treatment of the disclosure will inhibit or delay progress of baldness. An effective amount of said active ingredient solution given within the framework of the hair loss treatment of the disclosure is an amount sufficient to induce a positive benefit, for example, hair growth or mitigate an itchy scalp or flaky scalp, but low enough to avoid serious side effects, i.e., to provide a reasonable benefit to risk ratio, within the scope of sound judgment of the skilled artisan

The Betula Pubescens Flower Bud, Castanea Vesca, Phosphorus, and Populus Nigra Leaf Bud, extracts thereof, or portions thereof, can each be made into a liquid solution prior to combining to form the hair growth composition. The RNA can be made from fungus, and can be in power form prior to adding to the hair growth composition. The at least one human hair strand can be a full and intact strand of human hair that is soaked or submerged in a high-proof alcohol solvent. High-proof alcohol can be an alcohol solvent comprising at least 50% alcohol by volume.

### II. Compositions of the Application

Accordingly, in one aspect, the present application relates to a hair growth composition comprising:
a. Betula Pubescens Flower Bud;
b. Populus Nigra Leaf Bud;
c. at least one human hair strand; and
d. RNA.

Accordingly, in one aspect, the present application relates to a hair growth composition comprising:
a. Betula Pubescens Flower Bud with diluted Centisemal levels 1×, 2X , or 3X;
b. Castanea Vesca with diluted Centisemal levels 1×, 2X , or 3×
c. Human hair with diluted Centisemal levels 3C, 4C, 5C, 6C, 7C, or 8C
d. Phosphorous with diluted Centisemal levels 4×, 5×, 6×, 7×, 8×, 9×, 11×, 12×, 2C, 3C, 4C, 5C, 6C, or 7C
e. Populus Nigra Leaf Bud with diluted Centisemal levels 1×, 2X , or 3X;
f. RNA with diluted Centisemal levels 2×, 3X, 4X , or 5X ; and
g. Inactive ingredients wherein the inactive ingredients may be chosen from the group comprised of alcohol and water. In a further embodiment, the alcohol shall be 15% to 25% by volume.

Accordingly, in another aspect, the present application relates to a hair growth composition comprising:
a. Betula Pubescens Flower Bud having a Centisemal level of 2×;
b. Castanea Vesca having a Centisemal level of 2×;
c. Human hair having a Centisemal level of 4C or 7C;
d. Phosphorous having a Centisemal level of 5×, 6×, 8×, 12×, 3C, 5C, 6C, or 7C
e. Populus Nigra Leaf Bud having a Centisemal level of 2×;
f. RNA having a Centisemal level of 3X or 4X ; and
g. Inactive ingredients wherein the inactive ingredients may be chosen from the group comprised of alcohol and water. In a further embodiment, the alcohol shall be 17% to 22% by volume.

In another aspect, the present application relates to a method of topically administering the hair growth to a subject in need thereof:
a. topically applying the hair growth composition of the present application to the subject;

Accordingly, in another aspect, the present application relates to a hair growth composition comprising:
a. Human hair having a Centisemal level of 4C or 7C;
b. Inactive ingredients wherein the inactive ingredients may be chosen from the group comprised of alcohol and water. In a further embodiment, the alcohol shall be 18% to 21% by volume.

In another aspect, the present application relates to a method of topically administering the hair growth composition to a subject in need thereof:
a. topically applying the hair growth composition of the present application to the subject.

Accordingly, in another aspect, the present application relates to a hair growth composition comprising:
a. Betula Pubescens Flower Bud having a Centisemal level of 2×;
b. Inactive ingredients wherein the inactive ingredients may be chosen from the group comprised of alcohol and water. In a further embodiment, the alcohol shall be 20% by volume.

In another aspect, the present application relates to a method of sublingually administering the hair growth composition to a subject in need thereof:
a. applying a hair regrowth composition of the present application to the subject orally or under the subject's tongue.

In some aspects of the present disclosure, an effective amount of the hair growth composition can be administered or used by topical application to the body, for example, on the skin, scalp, hair, and/or another anatomical surface. The body can be either wet or dry when the composition is administered or used topically. Optionally, the body may be wetted with water prior to the administration of an effective amount or use of the hair growth composition. An effective amount of the hair growth may be administered by impregnating a patch or thin film apparatus and temporarily adhering the patch or thin film apparatus to the skin. An effective amount of the hair growth composition may be administered sublingually. An effective amount of the hair growth composition may be administered sublingually and topically.

In some embodiments, the hair growth composition may be a drug, cosmetic, and/or homeopathic product. In some embodiments, the hair growth composition may be a prescription or over-the-counter product. In other embodiments, an effective amount of the composition can be administered in water, on tiny sugar pellets, or in tablet form.

Topically administering an effective amount of the composition to the scalp can be performed by spraying or misting the composition directly to the scalp, skin, or body part where hair growth is desired. Topical administration does not result in existing hair appearing greasy or dirty after administration of the hair growth composition.

Sublingually administering an effective amount of the composition can be performed by spraying, misting, or dropping an effective amount of the hair growth composition under the tongue. Administration by dropping can be performed by using an eye dropper to administer the hair growth serum by dropping an effective amount of the hair growth serum under the tongue by using an eye dropper or similar device.

In some embodiments, the hair growth composition may comprise additives such as fragrance, taste modifiers such as bitter blockers and/or sweeteners, and various vitamins and minerals such as biotin, B vitamins, or other additives that are known to support hair growth and do not reduce the effectiveness of the active ingredients described above. In some embodiments, the hair growth composition may be a serum.

Since many modifications, variations, and changes in detail can be made to the described preferred embodiments of the disclosure, it is intended that all matters in the foregoing description and shown in the accompanying drawings be interpreted as illustrative and not in a limiting sense.

Although some, and maybe preferred, embodiments of the disclosure have been described in detail herein, those skilled in the art will recognize that various substitutions and modifications may be made without departing from the scope and spirit of the appended claims. Thus, the scope of the invention should be determined by the appended claims and their legal equivalents.

I claim:

1. A hair growth composition comprising:
a. Betula Pubescens Flower Bud;
b. Populus Nigra Leaf Bud;
c. at least one human hair strand; and
d. RNA.

2. The hair growth composition of claim 1, wherein the Betula Pubescens Flower Bud has a Centisemal level of 1X to 3X.

3. The hair growth composition of claim 1, further comprising at least one inactive ingredient of water, alcohol, or a combination thereof.

4. The hair growth composition of claim 1, wherein the RNA is extracted from fungus.

5. The hair growth composition of claim 1, further comprising phosphorus.

6. The hair growth composition of claim 1, wherein the hair growth composition is a liquid.

7. The hair growth composition of claim 1, wherein the hair growth composition is a serum.

8. A method growing hair in a subject in need of such treatment comprising administering the hair growth composition of claim 1, to a subject on an area where hair growth is desired sublingually, topically, by thin film patch, in tablet form, or a combination thereof.

9. The method of claim 8, wherein the administering of the hair growth composition of claim 1 topically comprises applying the hair growth composition of claim 1 to a scalp, wherein the hair growth composition does not cause existing hair on the scalp to feel or appear greasy.

10. A hair growth composition comprising:
a. Betula Pubescens Flower Bud;
b. Castanea Vesca;
c. at least one human hair soaked in an alcohol solvent of at least 50% alcohol by volume;
d. phosphorous;
e. Populus Nigra Leaf Bud;
f. RNA; and
g. at least one inactive ingredient.

11. The hair growth composition of claim 10, wherein the hair growth composition is a liquid.

12. The hair growth composition of claim 10, wherein the hair growth composition is a serum.

13. The hair growth composition of claim 10, wherein the hair growth composition is administered sublingually, topically, by thin film patch, in tablet form, or a combination thereof.

14. A hair growth composition comprising:
a. Betula Pubescens Flower Bud having a Centisemal level of 1X to 3X;
b. Castanea Vesca having a Centisemal level of 1X to 3X;
c. at least one human hair having a Centisemal level of 4C or 7C;
d. phosphorous having a Centisemal level of 5X or 5C, 6C, or 7C
e. Populus Nigra Leaf Bud having a Centisemal level of 1X to 3X;
f. RNA having a Centisemal level of 3X or 4X ; and
g. at least one inactive ingredient, wherein the inactive ingredient is alcohol, water, or a combination thereof.

15. The hair growth composition of claim 14, wherein the hair growth composition is a serum.

16. The hair growth composition of claim 14, wherein the hair growth composition is administered sublingually, topically, by thin film patch, in tablet form, or a combination thereof.

17. The hair growth composition of claim 16, wherein the tablet form is a sugar tablet and the hair growth composition is a liquid applied to the sugar tablet before ingesting.

18. The hair growth composition of claim 14, wherein the inactive ingredient comprises 5% to 30% of alcohol by volume of the hair growth composition.

19. The hair growth composition of claim 14, wherein the inactive ingredient is a combination of alcohol and water and comprises:
10% to 25% of alcohol by volume of the hair growth composition; and
the water is distilled water.

20. A method of treating itchy scalp and flaking on a subject in need of such treatment comprising administering the hair growth composition of claim 19 to the subject sublingually, topically, by thin film patch, in tablet form, or a combination thereof.
